(19) United States
(12) Patent Application Publication
(10) Pub. No.: US 2022/0192674 A1
(43) Pub. Date: Jun. 23, 2022

(54) ACTUATION STRUCTURE OF BLOOD VESSEL CLIP APPLIER

(71) Applicant: MEDSCOPE BIOTECH CO., LTD., Zhunan Town, Miaoli County (TW)

(72) Inventors: Shin-Hao HUANG, Zhudong Town (TW); Chen-Xuan WU, Hsinchu City (TW)

(73) Assignee: MEDSCOPE BIOTECH CO., LTD., Zhunan Town, Miaoli County (TW)

(21) Appl. No.: 17/600,769

(22) PCT Filed: Mar. 31, 2020
(86) PCT No.: PCT/CN2020/082541

§ 371 Date: Oct. 1, 2021

(30) Foreign Application Priority Data
Apr. 3, 2019 (CN) 201910266128.5

(51) Int. Cl. A61B 17/128

(52) U.S. Cl. CPC A61B 17/128; A61B 2017/00367

(57) ABSTRACT

Disclosed is an actuation structure of a blood vessel clip applier, which is driven by an operating unit to make a jaw clamp a clip. The actuation structure of the blood vessel clip applier includes a body with a bump; a tube set connected to the body; a loading push rod capable of pushing the clip forward; a bolt axially and movably arranged on the body; a feeding loop claw pivoted at the bolt and has a front end and a rear end; a loading plug arranged on the body and linked to the loading push rod; a loading spring for pushing the loading plug to move backward; a recoil push rod so configured to push the recoil push rod forward to drive the jaw clamp the clip; and a recoil spring for pushing the recoil push rod to move backward.

### BACKGROUND OF THE INVENTION

### 1. Field of the Invention

The present invention is related to blood vessel clip applier technology, especially an actuation structure of a blood vessel clip applier.

### 2. Description of the Related Art

The known actuation structure of a blood vessel clip applier has a more complicated transmission mechanism, so its transmission is less reliable, and may cause overlapping actions or neutral gears. Therefore, the lack of the existing actuation structure of a blood vessel clip applier still needs to be improved.

### SUMMARY OF THE INVENTION

The present invention has been accomplished under the circumstances in view. It is the main object of the present invention to provide an actuation structure of a blood vessel clip applier, which can simplify the transmission mechanism, make the transmission action more reliable, and the action is smoother.

To achieve the object stated above, the present invention provides an actuation structure of a blood vessel clip applier driven by an operating unit to make a jaw clamp a clip. The actuation structure of the blood vessel clip applier comprises a body comprising a bump; a tube set connected to the body; a loading push rod movably set in said tube set back and forth for pushing the clip forward; a bolt axially movably arranged on the body; a feeding loop claw pivoted to the bolt, the feeding loop claw comprising a front end and a rear end so configured that when the feeding loop claw moves forward, the feeding loop claw is touched by the bump of the body so that the front end of the feeding loop claw changes from a tilted out position to a lower pressure position; a loading plug set in the body to link the loading push rod; a loading spring o push the loading plug to move backward; wherein when the front end of the feeding loop claw is tilted out, the loading plug is pushed forward; when the front end of the feeding loop claw is pressed down and kept away from the loading plug, the loading spring pushes the loading plug back to the original position; a recoil push rod movably set in the tube set, so that after the loading plug returns to the original position, the front end of the feeding loop claw is still depressed to push the recoil push rod forward to drive the jaw clamp the clip; and a recoil spring) used to push the recoil push rod to move backward.

Through the structure and the action process, it can simplify the transmission mechanism, make the transmission action more reliable, and the action is smoother, so that the purpose of the invention is achieved.

Preferably, the bolt is provided with a receiving slot, and the feeding loop claw is installed in the receiving slot.

Preferably, the body comprises a bushing, and the loading plug is installed in the bushing.

Preferably, the actuation structure of the blood vessel clip applier further comprises an elastic piece set on said bolt. The elastic piece can be pressed against the rear end of the feeding loop claw so that the front end of the feeding loop claw is tilted out against the loading plug.

Preferably, the operating unit comprises a bolt link pivoting the bolt, and the pivoting center of the bolt link and the bolt is axially behind the pivoting center of the feeding loop claw and the bolt.

Preferably, the loading plug surrounds the recoil push rod.

### BRIEF DESCRIPTION OF THE DRAWINGS

FIG. 1 is an oblique top elevational view of a blood vessel clip applier of the preferred embodiment of the present invention.

FIG. 2 is a sectional elevational side view of the blood vessel clip applier of the preferred embodiment of the present invention.

FIG. 3 is another sectional elevational side view of the blood vessel clip applier of the preferred embodiment of the present invention.

FIG. 4 is a sectional elevational side view of a part of the blood vessel clip applier of the preferred embodiment of the present invention.

FIG. 5 is an oblique top elevational view of the bolt of the blood vessel clip applier of the preferred embodiment of the present invention.

FIG. 6 is an oblique top elevational view of the loading push rod, the loading plug and the loading spring of the bolt of the blood vessel clip applier of the preferred embodiment of the present invention.

FIG. 7 is an oblique top elevational view of the recoil push rod and the recoil spring of the bolt of the blood vessel clip applier of the preferred embodiment of the present invention.

FIG. 8 is an explanatory diagram of the preferred embodiment of the present invention not yet operating state.

FIG. 9 is an explanatory diagram of the preferred embodiment of the present invention just pushed state.

FIG. 10 is a schematic diagram of the preferred embodiment of the present invention, wherein the loading plug has just been pushed back by the loading spring.

FIG. 11 is an explanatory diagram of the preferred embodiment of the present invention where the recoil push rod is pushed forward by the bolt.

FIG. 12 is a three-dimensional exploded view of the front elements of the preferred embodiment of the present invention.

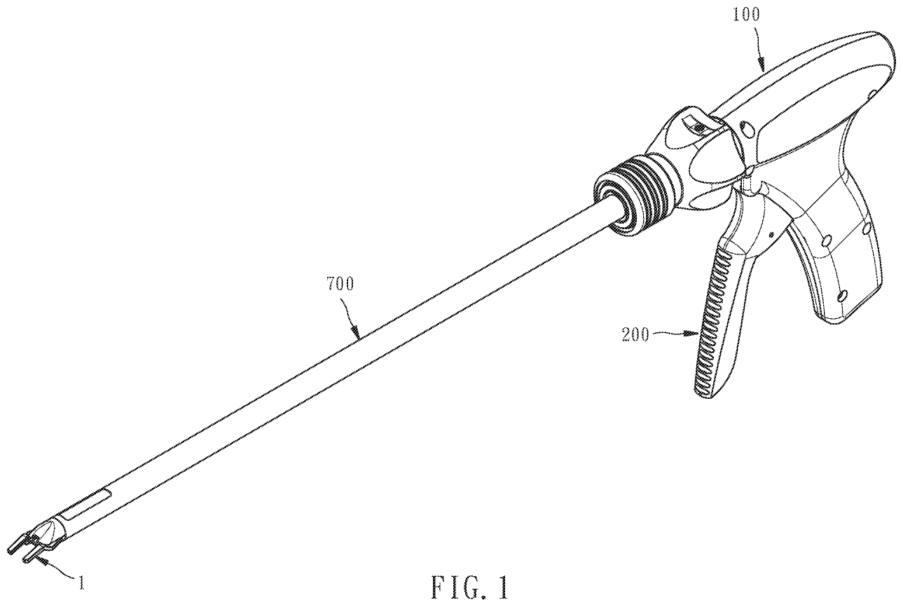

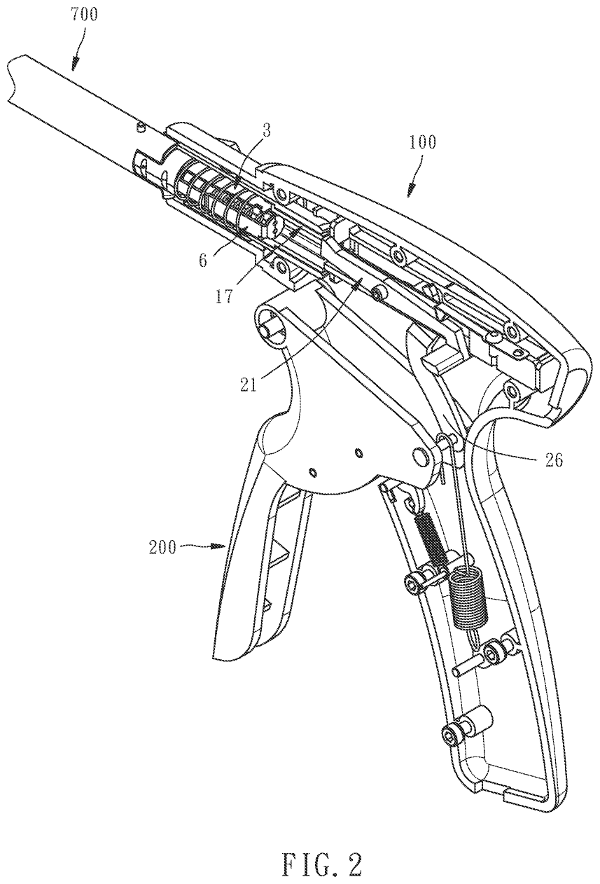

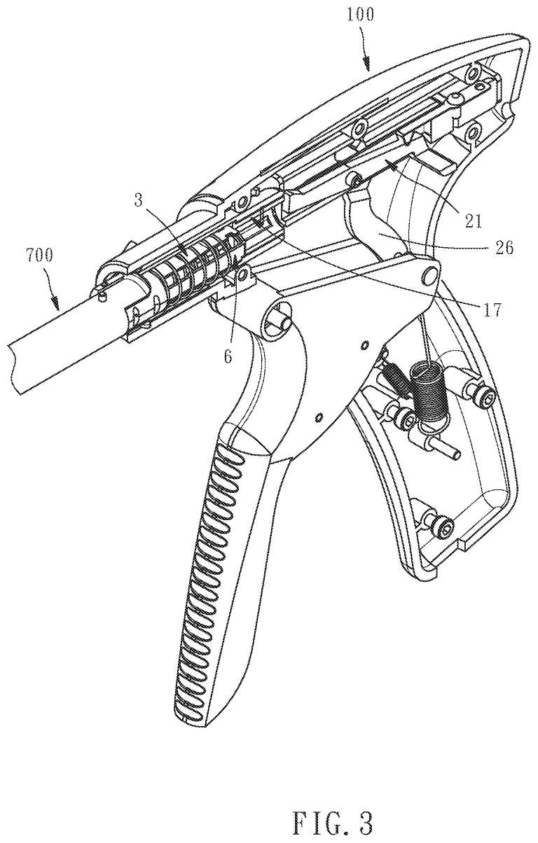

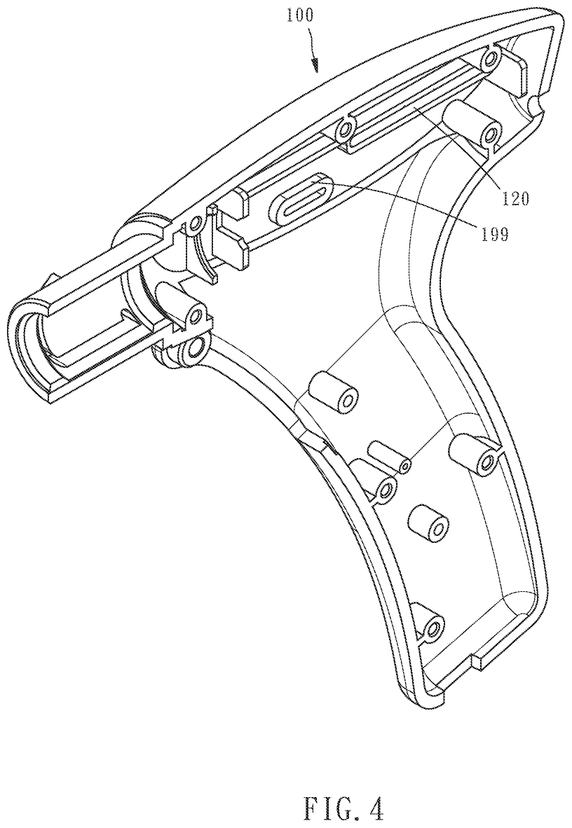

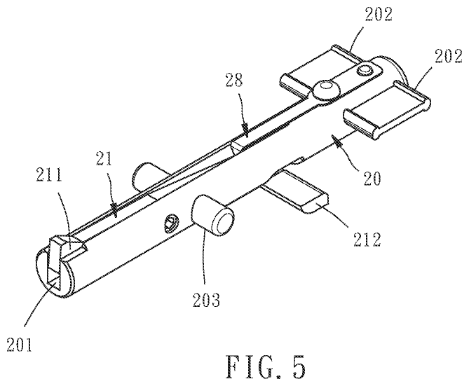

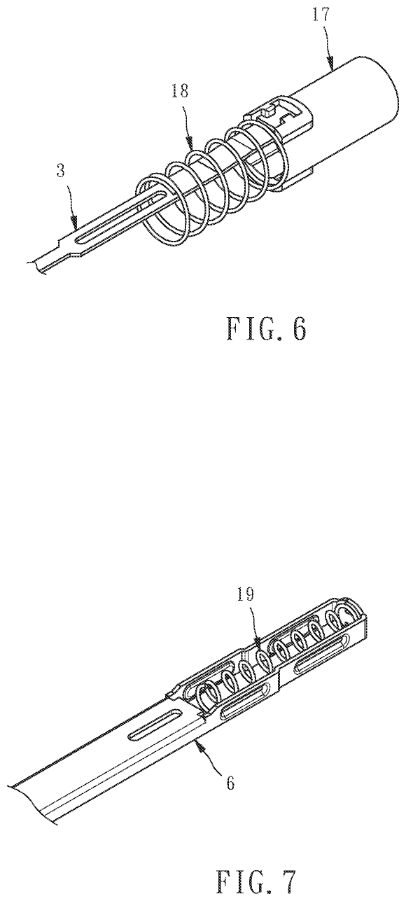

### DETAILED DESCRIPTION OF THE INVENTION

The present invention will be further described below in conjunction with the accompanying drawings and the preferred embodiment:

The actuation structure of a blood vessel clip applier provided by the present invention is driven by an operating unit 200 to make a jaw 1 clamp a clip 35.

The actuation structure of the blood vessel clip applier comprises:

a body100 comprising a bushing 110, a sliding groove 120 located on each of left and right sides of a rear of two shells thereof, and a bump 199 located on each of left and right sides of a middle section of the two shells;

a tube set 700 connected to the body 100;

a loading push rod 3 movably set on the tube set 700 back and forth to push the clip 35 forward;

a bolt 20 axially movably arranged on the body100, the bolt 20 comprising a receiving slot 201 in a front thereof, a sliding wing 202 located on each of left and right sides of a rear thereof and respectively slidingly fitted to the sliding grooves 120 of the body 100, and a pivot rod 203 located on each of left and right sides of a middle section thereof for the operating unit 200 to pivotally push the bolt 20;

a feeding loop claw 21 accommodated in the receiving slot 201 of the bolt 20 and pivotally connected to the bolt 20, in more detail, the feeding loop claw 21 being pivotally connected to the bolt 20 and capable of being tilted to a tilted out position and a lower pressure position, the feeding loop claw 21 having a front end 211 and a rear end 212 so configured that when the feeding loop claw 21 moves forward, its rear end 212 is pressed by the bump 199 so that the feeding loop claw 21 reaches the lower pressure position;

an elastic piece 28 arranged on the bolt 20, and the elastic piece 28 being pressable against the rear end 212 of the feeding loop claw 21 so that the front end 211 of the feeding loop claw 21 is lifted out and abuts against a loading plug 17, in more detail, the elastic piece 28 having a top end that can be pressed against the rear end 212 of the feeding loop claw 21 so that the front end 211 of the feeding loop claw 21 is located at the tilted out position;

wherein, the operating unit 200 has a bolt link 26 pivoting the bolt 20, and the pivot center of the bolt link 26 and the bolt 20 is axially behind the pivot center of the feeding loop claw 21 and the bolt 20;

a loading plug 17 installed in the body 100 to link the loading push rod 3; in this embodiment, the loading plug 17 being installed in the bushing 110;

a loading spring 18 used to push the loading plug 17 and move backward;

wherein when the front end 211 of the feeding loop claw 21 is tilted out, the loading plug 17 can be pushed forward; when the front end 211 of the feeding loop claw 21 is pressed down and kept away from the loading plug 17, the loading spring 18 can push the loading plug 17 back to the original position;

a recoil push rod 6, which can be movably set in the tube set 700 back and forth and so configured that after the loading plug 17 returns to the original position, the front end 211 of the feeding loop claw 21 is still depressed and can continue to push the recoil push rod 6 forward to drive the jaw 1 to clamp the clip 35; and

a recoil spring 19 used to push the recoil push rod 6 and move backward; wherein, the loading plug 17 can surround the recoil push rod 6.

The various operating states of the present invention are described as follows:

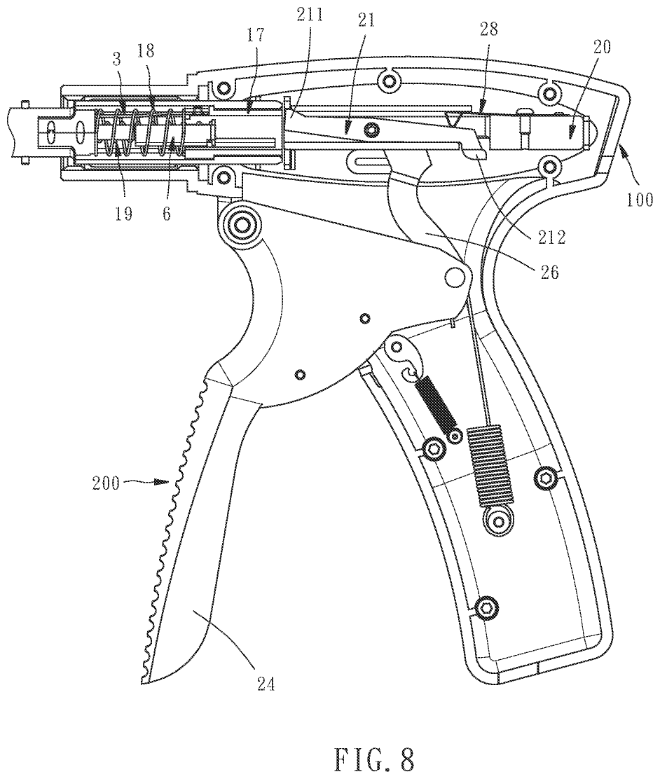

As shown in FIG. 8 (original state), where the operating unit 200 has not been operated, the bolt 20 is controlled by the operating unit 200 and is located at the rear of its axis, and the feeding loop claw 21 is pressed to protrude its front end 211, wherein, the loading plug 17 is pushed by the loading spring 18 and is located at its final position, and the recoil push rod 6 is pushed by the recoil spring 19 and is located at its final position.

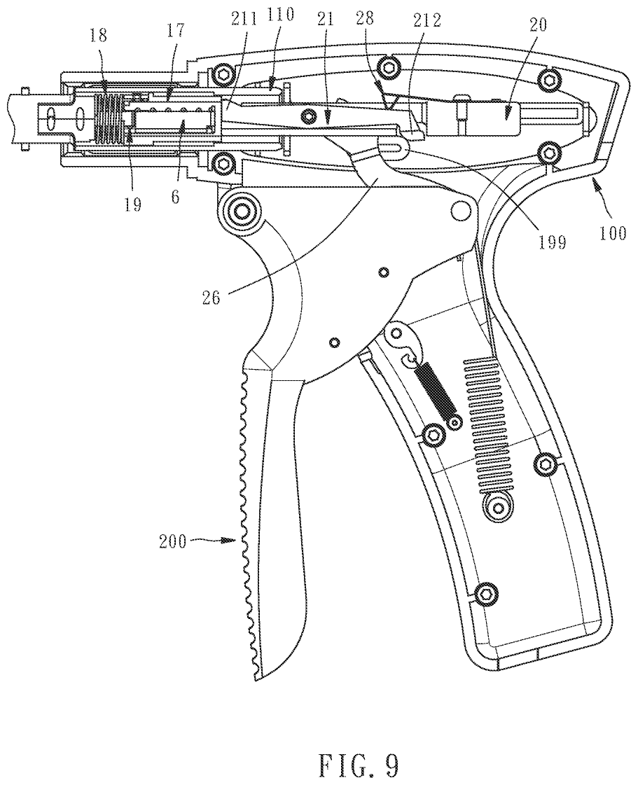

As shown in FIG. 9 (push state), start to operate the operating unit 200, that is, hold its handle 24, the operating unit 200 drives the bolt 20 to move forward axially, and the front end 211 of the feeding loop claw 21 pushes the loading plug 17 forward (overcomes the elastic resistance of the loading spring 18).

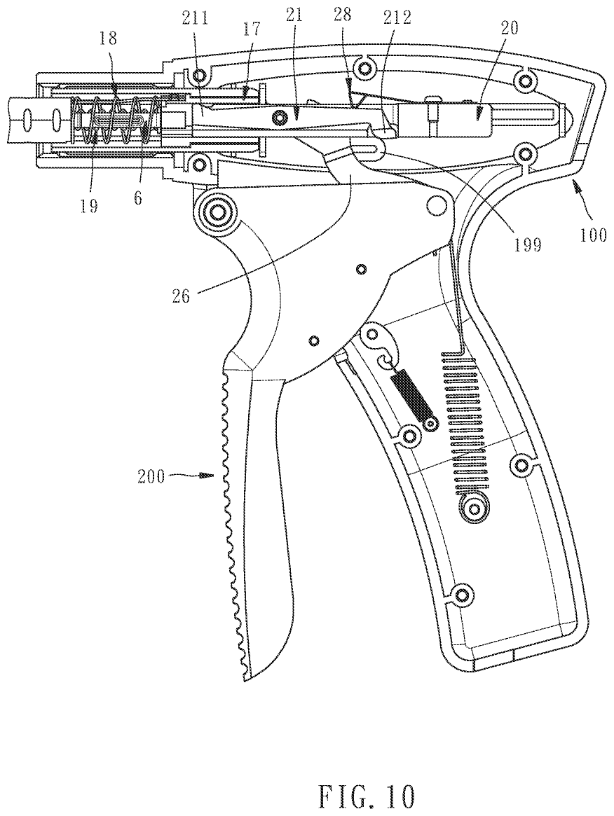

As shown in FIG. 10 (the front end 211 starts to be depressed), when the continuous operation advances, the feeding loop claw 21 deflects because its rear end 212 is touched by the bump 199 of the body 100, and the front end 211 of the feeding loop claw 21 no longer reaches the loading plug 17, and the loading plug 17 is then pushed back by the loading spring 18 and returns to its original final position, as shown in FIG. 10, that is, the loading plug 17 has just returned to the state.

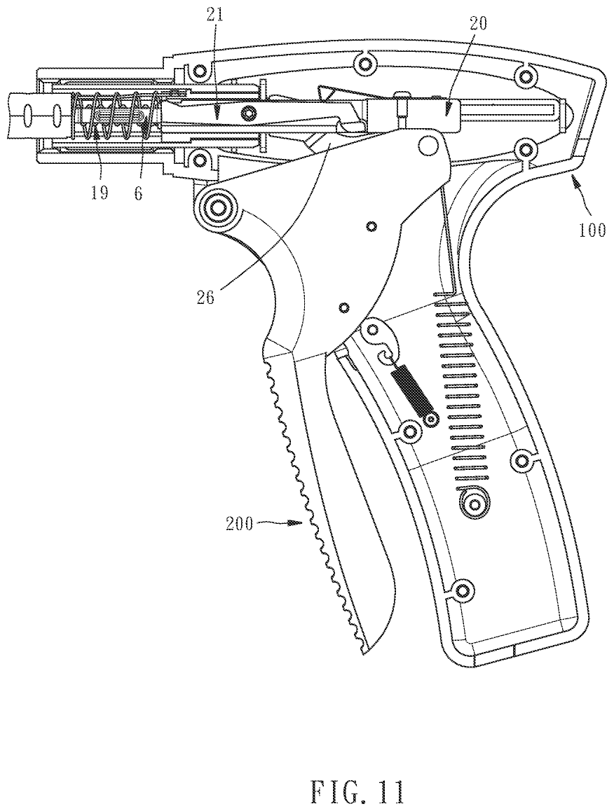

As shown in FIG. 11 (clip 35 clamping state), continue to operate the operating unit 200 again, the bolt 20 is driven by the operating unit 200 to continue to move forward to push the recoil push rod 6 to overcome the elastic force of the recoil spring 19, and to make the recoil push rod 6 move forward, so that the recoil push rod 6 can drive the jaw 1 to clamp the clip 35 in it.

Then, release the operating unit 200 without operating, and each component returns to the original state, as shown in FIG. 8.

Through the structure and the action process, it can simplify the transmission mechanism, make the transmission action more reliable, and the action is smoother, so that the purpose of the invention is achieved.

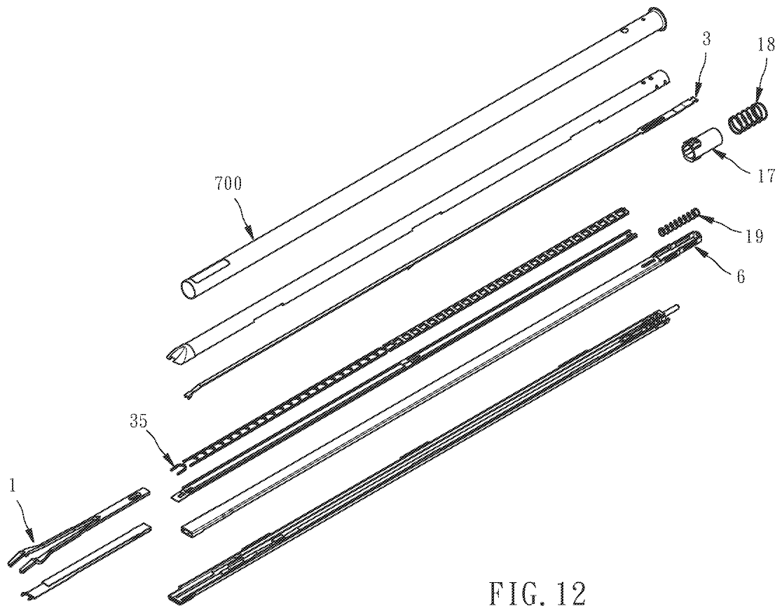

As shown in FIG. 12, a three-dimensional exploded view of the front elements of the preferred embodiment of the present invention is shown, wherein the unexplained structure and principle of action can be generally the same as the invention patent of Taiwan Announcement I627933, which will not be described in detail here. However, the front assembly of the present invention is not limited to only include the structure and principle of action, and can also be of various other types.

For example, the bolt link 26 is not limited to two as described above, and it can also be only one.

Or, the rear end 212 of the feeding loop claw 21 is not necessarily located at the rear, as long as it can be cooperated to activate the feeding loop claw 21.

In summary, the actuation structure of a blood vessel clip applier of the present invention can indeed simplify the transmission mechanism, make the transmission action more reliable, and the action is smoother, so it does achieve the stated purpose of the invention.

Industrial applicability:

The actuation structure of a blood vessel clip applier of the present invention can indeed simplify the transmission mechanism, make the transmission action more reliable, and the action is smoother, so it does achieve the purpose of the invention.

Of course, the present invention can also have other various embodiments. Without departing from the spirit and essence of the present invention, those skilled in the art can make various corresponding changes and modifications according to the present invention, but these corresponding changes and modifications all should belong to the protection scope of the claims of the present invention.

1. An actuation structure of a blood vessel clip applier driven by an operating unit (200) to make a jaw (1) clamp a clip (35), the actuation structure of the blood vessel clip applier comprising:
a body (100) comprising a bump (199);
a tube set (700) connected to said body (100);
a loading push rod (3) movably set in said tube set (700) back and forth for pushing said clip (35) forward;
a bolt (20) axially movably arranged on said body (100);
a feeding loop claw (21) pivoted to said bolt (20), said feeding loop claw (21) comprising a front end (211) and a rear end (212) so configured that when said feeding loop claw (21) moves forward, said feeding loop claw (21) is touched by said bump (199) of said body (100) so that the front end (211) of said feeding loop claw (21) changes from a tilted out position to a lower pressure position;
a loading plug (17) set in said body (100) to link said loading push rod (3);
a loading spring (18) to push said loading plug (17) to move backward;
wherein when the front end (211) of said feeding loop claw (21) is tilted out, said loading plug (17) is pushed forward; when the front end (211) of said feeding loop claw (21) is pressed down and kept away from said loading plug (17), said loading spring (18) pushes said loading plug (17) back to the original position;
a recoil push rod (6) movably set in said tube set (700), so that after said loading plug (17) returns to the original position, the front end (211) of said feeding loop claw (21) is still depressed to push said recoil push rod (6) forward to drive said jaw (1) clamp said clip (35); and
a recoil spring (19) used to push said recoil push rod (6) to move backward.

2. The actuation structure of the blood vessel clip applier as claimed in claim 1, wherein said bolt (20) comprises a receiving slot (201), and said feeding loop claw (21) is installed in said receiving slot (201).

3. The actuation structure of the blood vessel clip applier as claimed in claim 2, wherein said body (100) comprises a bushing (110), and said loading plug (17) is installed in said bushing (110).

4. The actuation structure of the blood vessel clip applier as claimed in claim 1, wherein said body (100) comprises a bushing (110), and said loading plug (17) is installed in said bushing (110).

5. The actuation structure of the blood vessel clip applier as claimed in claim 1, further comprising an elastic piece (28) set on said bolt (20), said elastic piece (28) being pressable against the rear end (212) of said feeding loop claw (21) so that the front end (211) of said feeding loop claw (21) is tilted out against said loading plug (17).

6. The actuation structure of the blood vessel clip applier as claimed in claim 5, wherein said operating unit (200) comprises a bolt link (26) pivoting said bolt (20), and the pivoting center of said bolt link (26) and said bolt (20) is axially behind the pivoting center of said feeding loop claw (21) and said bolt (20).

7. The actuation structure of the blood vessel clip applier as claimed in claim 1, wherein said operating unit (200) comprises a bolt link (26) pivoting said bolt (20), and the pivoting center of said bolt link (26) and said bolt (20) is axially behind the pivoting center of said feeding loop claw (21) and said bolt (20).

8. The actuation structure of the blood vessel clip applier as claimed in claim 7, wherein said loading plug (17) surrounds said recoil push rod (6).

9. The actuation structure of the blood vessel clip applier as claimed in claim 5, wherein said loading plug (17) surrounds said recoil push rod (6).

10. The actuation structure of the blood vessel clip applier as claimed in claim 1, wherein said loading plug (17) surrounds said recoil push rod (6).
